(19) United States
(12) Patent Application Publication
(10) Pub. No.: US 2023/0380609 A1
(43) Pub. Date: Nov. 30, 2023

(54) REACTIVE PILLOW AND THE METHOD OF FABRICATION

(71) Applicant: Super Rich Moulders Limited, Hong Kong (HK)

(72) Inventors: Li FU, Hong Kong (HK); Yam CHONG, Hong Kong (HK); Chun Wah CHU, Hong Kong (HK); Chenmin LIU, Hong Kong (HK)

(21) Appl. No.: 18/249,547

(22) PCT Filed: Oct. 8, 2021
(86) PCT No.: PCT/CN2021/122537

§ 371 Date: Apr. 19, 2023

Related U.S. Application Data
(60) Provisional application No. 63/198,436, filed on Oct. 19, 2020.

(51) Int. Cl. A47G 9/10

(52) U.S. Cl. CPC A47G 9/1027; A47G 9/1081

(57) ABSTRACT

Provided herein is a reactive pillow comprising: a pressure sensor mat (11) including a first electrode fabric layer (20) having a plurality of first conductive portions (13) and a plurality of first non-conductive portions; a second electrode fabric layer (21) having a plurality of second conductive portions (14) and a plurality of second non-conductive portions; and a piezoresistive fabric layer (24) having a sheet resistance of at least 50K ohm/square; an actuator (40) comprising a plurality of first airbags (41, 42, 43) and a second airbag (44), each of the airbags (41, 42, 43, 44) having an inflated configuration corresponding to an increase in the volume of the airbags (41, 42, 43, 44) and a deflated configuration corresponding to a decrease in the volume of the airbags (41, 42, 43, 44); a controller communicating with the pressure sensor mat (11) and the actuator (40) communicating with the controller to actuate the controller to convert the airbags (41, 42, 43, 44) between the inflated configuration and the deflated configuration thereof.

### CROSS-REFERENCE TO RELATED APPLICATIONS

The present application claims priority from a U.S. provisional patent application No. 63/198,436 filed Oct. 19, 2020, and the disclosure of which is incorporated herein by reference in its entirety.

### FIELD OF THE INVENTION

The present invention provides a supporting means, in particular pillows which can automatically adjust height and shape responsive to the contact pressure recorded from time to time through a flexible pressure sensor mat, and method for fabricating the same.

### BACKGROUND

Sleeping is one of the most essential activities and plays a vital role in good health and well-being in human lives. Comfortable bedding, especially pillow, is important for health and quality of life. It is estimated that neck pain or discomfort occurs in nearly a quarter of the population, and poor pillow design is apparently the main cause of this extremely high incidence. Many researchers tried to evaluate comfortableness of pillows with those factors such as stiffness, shape and thermal properties. Both shape and stiffness of pillows will affect the support of neck and head during sleeping. Improper pillow support has adverse effects on the cervical spine, leading to neck pain and cervicogenic headache. Current pillow products in the market usually have fixed shape and stiffness, which cannot be adjusted after purchase. However, side sleepers, back sleepers and stomach sleepers may need a pillow with different stiffness and height to keep head and neck in alignment with spine. Meanwhile, pillow selection also depends on mattress firmness. A comfortable pillow requires shape transformation to fit the appropriate sleeping posture for each user. Even though there is mattress product that can monitor pressure distribution during sleeping, active adjustment based on the signal from sensor is rare in the market. There is no pillow product that can address the supporting issue specific to the user and the sleeping posture.

The comfortableness of a pillow can be characterized by the pressure at the contact area where the user sleeps on it, therefore it requires a matrix of pressure sensors to detect the pressure at each contact area and monitor the pressure distribution across the whole pillow surface. Traditionally, pressure sensors were individual gauges made from metal or semiconductors. These gauges are usually rigid and required to be attached to beams to convert load to strain. They can monitor small area with high accuracy but the integration will be complicated and expensive. These sensors are not suitable for applying in the pillow due to the rigid form. Flexible pressure sensors include unique advantages of flexibility and low cost. They are emerging as promising candidate for many applications, including but not limited to touching sensing, large area pressure monitoring and mapping, gait analysis and sports scoring. Thin and flexible sensors printed on plastic foil can monitor pressure over a wide range, and it requires a smooth surface (flat or curved) to support the sensors during their operation. However, as for pillow application, the contact surface is usually non-flat with random 3D shape. Creases formed on sensors will result in reliability issue since the printed material cannot withstand repeated shear force during contact. Furthermore, the plastic foil substrate is not breathable and may let user feel uncomfortable.

In view of the disadvantages of the existing adjustable pillow product, there is a need for providing a reactive pillow system which is not only able to adjust height and adapt the shape of the pillow according to the contact pressure applied by the user.

### SUMMARY OF THE INVENTION

Accordingly, a first aspect of the present invention provides a reactive pillow. The reactive pillow comprises a pressure sensor mat, an actuator and a controller. The pressure sensor mat comprises a first electrode fabric layer, a second electrode fabric layer and a piezoresistive fabric layer. The first electrode fabric layer includes a plurality of first conductive portions and a plurality of first non-conductive portions where the first conductive portions are interlaced with the first non-conductive portions. The second electrode fabric layer includes a plurality of second conductive portions and a plurality of second non-conductive portions where the second conductive portions are interlaced with the second non-conductive portions. The piezoresistive fabric layer has a sheet resistance of at least 50K ohm/square and is configured to engage with the first electrode fabric layer and the second electrode fabric to form the pressure sensor mat. In addition, the actuator comprises a plurality of first airbags and a second airbag. The first airbags are positioned on the second airbag and each of the airbags has an inflated configuration corresponding to an increase in the volume of the airbags and a deflated configuration corresponding to a decrease in the volume of the airbags. The controller communicates with the pressure sensor mat and the actuator communicates with the controller to actuate the controller to convert the airbags between the inflated configuration and the deflated configuration thereof.

In one embodiment of the first aspect of the present invention, there is provided a reactive pillow where the first conductive portions and the second conductive portions are aligned perpendicularly.

In one embodiment of the present invention, there is provided a reactive pillow where the first and second conductive portions are woven or knitted fabrics made of or comprising conductive yarns.

In another embodiment of the present invention, there is provided a reactive pillow where the first and second non-conductive portions are woven or knitted fabrics made of or comprising non-conductive yarns.

In another embodiment of the present invention, there is provided a reactive pillow where each of the first and second conductive portions has a width of approximately 3 mm to 10 mm.

In another embodiment of the present invention, there is provided a reactive pillow where each of the first and second non-conductive has a width of approximately 5 mm to 50 mm.

In another embodiment of the present invention, there is provided a reactive pillow where the piezoresistive fabric layer is woven or knitted fabric made of or comprising semi-conductive yarn.

In another embodiment of the present invention, there is provided a reactive pillow where the piezoresistive fabric layer further comprises a non-conductive fabric and a plurality of filling portions with a piezoresistive ink.

In another embodiment of the present invention, there is provided a reactive pillow where the piezoresistive ink comprises a polymer, a conductive material and a solvent.

In another embodiment of the present invention, there is provided a reactive pillow where the polymer has a concentration approximately from 1% to 10% by weight, the conductive material has a concentration approximately from 0.1% to 2% by weight and the solvent has a concentration approximately from 90% to 95% by weight.

In another embodiment of the present invention, there is provided a reactive pillow where the sheet resistance of the filling portions is at least 50K ohm/square.

In another embodiment of the present invention, there is provided a reactive pillow where the filling portions have one or more shapes being selected from circle, square, and rectangle, with a width of approximately 5 mm to 15 mm and a space approximately from 3 mm to 50 mm.

In another embodiment of the present invention, there is provided a reactive pillow where the actuator further comprises at least one micro pump, at least one tube and at least one valve, and are configured to convert the airbags between the inflated configuration and the deflated configuration.

In another embodiment of the present invention, there is provided a reactive pillow where the first airbags are configured to convert between inflated configuration and the deflated configuration such that the relative volume corresponding to the left, right, top bottom of the pillow can be adapted.

In another embodiment of the present invention, there is provided a reactive pillow where the first airbags comprise at least three airbags.

In another embodiment of the present invention, there is provided a reactive pillow where the second airbag is configured to convert between inflated configuration and the deflated configuration such that relative volume corresponding to the height of the pillow can be adapted.

In another embodiment of the present invention, there is provided a reactive pillow where the reactive pillow further comprises a Bluetooth module configured to communicate with a user terminal.

A second aspect of the present invention provides a method for fabricating a reactive pillow, which includes (1) providing a pressure sensor mat comprising a first electrode fabric layer having a plurality of first conductive portions and a plurality of first non-conductive portions where the first conductive portions are interlaced with the first non-conductive portions, a second electrode fabric layer having a plurality of second conductive portions and a plurality of second non-conductive portions where the second conductive portions are interlaced with the second non-conductive portions and a piezoresistive fabric layer having a sheet resistance of at least 50K ohm/square, the piezoresistive fabric layer being configured to engage with the first electrode fabric layer and the second electrode fabric; (2) providing an actuator comprising a plurality of first airbags and a second airbag, the first airbags being positioned on the second airbag, each of the airbags having an inflated configuration corresponding to an increase in the volume of the airbags and a deflated configuration corresponding to a decrease in the volume of the airbags; (3) providing a controller communicating with the pressure sensor mat and the actuator communicating with the controller to actuate the controller to convert the airbags between the inflated configuration and the deflated configuration. More specifically, the first conductive portions and the second conductive portions are aligned perpendicularly.

In one embodiment of the second aspect of the present invention, there is provided a method for fabricating a reactive pillow where the first and second conductive portions have a width of approximately 3 mm to 10 mm, and each of the first and second non-conductive has a width of approximately 5 mm to 50 mm.

In another embodiment of the second aspect of the present invention, there is provided a method for fabricating a reactive pillow where the piezoresistive ink comprises a polymer with a concentration approximately from 1% to 10% by weight, a conductive material with a concentration approximately from 0.1% to 2% by weight and a solvent with a concentration approximately from 90% to 95% by weight.

### BRIEF DESCRIPTION OF THE DRAWINGS

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 depicts a reactive pillow with integrated pressure sensor mat in one embodiment of the present invention.

FIG. 2 depicts a sensor array design with column-row structure in one embodiment of the present invention.

FIG. 3 is a schematic diagram showing the piezoresistive fabric with non-conductive textile and piezoresistive region in one embodiment of the present invention.

FIG. 4 is a schematic diagram depicting a flexible actuator with multi-zonal design in one embodiment of the present invention.

FIG. 5 is a schematic diagram showing the air path and control diagram for the actuator in one embodiment of the present invention.

FIG. 6 is a schematic diagram showing the sleeping posture adjustment by the flexible actuator in one embodiment of the present invention.

FIG. 7A depicts a flexible actuator with four actuation zones in one embodiment of the present invention.

FIG. 7B depicts a flexible actuator system with five actuation zones in one embodiment of the present invention.

FIG. 7C depicts a flexible actuator system with six actuation zones in one embodiment of the present invention.

FIG. 7D depicts a flexible actuator system with six actuation zones in another embodiment of the present invention.

FIG. 8 depicts a hardware control block diagram for the reactive pillow in one embodiment of the present invention.

FIG. 9 depicts a feedback control system block diagram for the reactive pillow in one embodiment of the present invention.

FIG. 10 depicts a flow chart showing control of pressurization and depressurization of an airbag in one embodiment of the present invention.

Skilled artisans will appreciate that elements in the figures are illustrated for simplicity and clarity and have not necessarily been depicted to scale.

### Definitions

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terms “a” or “an” are used to include one or more than one and the term “or” is used to refer to a nonexclusive “or” unless otherwise indicated. In addition, it is to be understood that the phraseology or terminology employed herein, and not otherwise defined, is for the purpose of description only and not of limitation. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Value in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a concentration range of “about 0.1% to about 5%” should be interpreted to include not only the explicitly recited concentration of about 0.1 wt. % to about 5 wt. %, but also the individual concentrations (e.g., 1%, 2%, 3%, and 4%) and the sub-ranges (e.g., 0.1% to 0.5%, 1.1% to 2.2%, and 3.3% to 4.4%) within the indicated range.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated.

### DETAILED DESCRIPTION

In the following description, the present reactive pillows are set forth as preferred examples. It will be apparent to those skilled in the art that modifications, including additions and/or substitutions may be made without departing from the scope and the spirit of the invention. Specific details may be omitted so as not to obscure the invention; however, the disclosure is written to enable one skilled in the art to practice the teachings herein without undue experimentation.

The present invention provides a reactive pillow which is able to detect the pressure at multiple locations simultaneously and adjust the shape and stiffness of the pillow accordingly so as to alleviate neck discomfort and chronic fatigue during sleeping. When a human sleep on a pillow, it was estimated that the contact pressure is roughly in the range of several kilo Pascal to a few dozens of kilo Pascal, depending on user and sleeping posture. In order to detect the contact pressure, a sensor array consisting of spaced sensor across the required surface can be fabricated as a pressure sensor mat to be positioned on top of or beneath the pillow. However, if the pressure sensor mat is placed beneath the pillow, the detected pressure is usually lower than that placed on top of pillow, mainly due to the dissipation of pressure by the increased contact area through the soft and deformable pillow. To acquire more accurate pressure measurement in the range that matches the sensitivity of piezoresistive sensors, the pressure sensor mat in the present invention is positioned onto the top surface of the pillow. After the collection of pressure data through the pressure sensor mat on the pillow, the change of the pillow shape and stiffness will rely on a flexible actuator to communicate with the pressure sensor mat for real-time adjustment. More specifically, the actuator is configured to adjust height and softness of the pillow based on the pressure detected. A flexible actuator with multi-actuation zones each comprising a plurality of airbags will allow users to adjust a tilt angle between neck and head, which could align the neck with the spine better and alleviate the discomfort caused by improper neck support. It is also applicable to check the breathing of a person sleeping on a pillow by monitoring the subtle difference of pressure during inhaling and exhaling. In addition, with a snore sensor integrated in the reactive pillow, the pillow is able to adjust the sleeping posture to control snoring and improve sleeping quality.

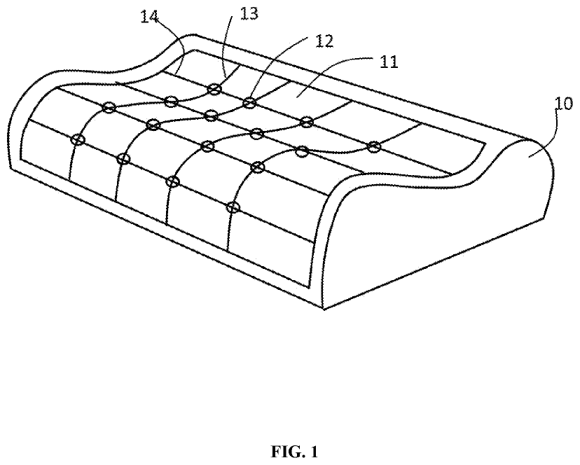

FIG. 1 illustrates a reactive pillow incorporated with a pressure sensor mat according to one embodiment of the present invention. The flexible pressure sensor mat 11 is positioned on top of pillow body 10. Each sensor is defined by each of the intersections 12 between conductive columns 13 and conductive rows 14.

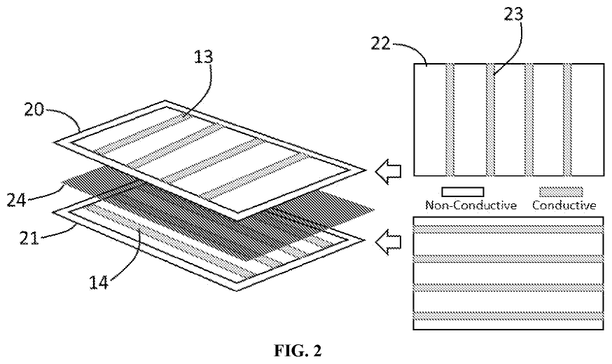

FIG. 2 shows a sensor array arranged as a column-row structure in the pressure sensor mat 11. Both top electrode sheet 20 and bottom electrode sheet 21 consist of non-conductive region 22 and conductive region 23, where the non-conductive region 22 are interlaced with the conductive region 23. The conductive region 23 includes one or more conductive columns 13 or conductive rows 14. The conductive columns 13 in top electrode sheet 20 and conductive rows 14 in bottom electrode sheet 21 extend in X and Y directions respectively. The width of the conductive columns 13 and the conductive rows 14 are approximately in the range of 5 mm to 10 mm, but can be tuned based on the application requirement. In addition, the conductive columns 13 or conductive rows 14 are usually spaced apart by the non-conductive region 22 with a space in a range approximately from 5 mm to 50 mm, depending on the resolution requirement of the pressure sensor mat. The crossing point or overlaying area where each of the conductive columns 13 intersects with each of conductive rows 14 is characterized as one sensor. Meanwhile, the piezoresistive layer 24 made of piezoresistive materials has high initial resistivity and is sandwiched between the top electrode sheet 20 and the bottom electrode sheet 21. The sheet resistance of the piezoresistive layer 24 is at least or preferably greater than 50 K ohm/square to minimize the crosstalk among the neighboring sensors when large area of the pressure sensor mat is pressed at the same time.

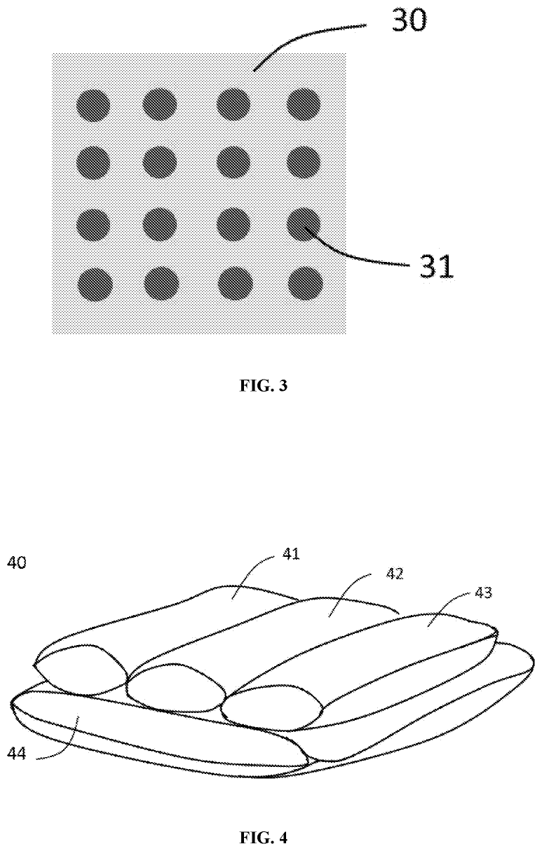

The piezoresistive layer 24 is a textile made of piezoresistive yarns. The piezoresistive yarns have high resistivity and can be woven or knitted into blank fabric without pattern. The resistivity of the piezoresistive layer will limit the sensitivity and sensing range of sensors, and the piezoresistive fabric could be, for example, but not limited to cotton fabric, blended fabric or synthetic fabric such as polyester or LYCRA for elasticity required in the present invention. In another embodiment of the present invention, the piezoresistive layer 24 includes non-conductive fabric 30 and piezoresistive ink coated in the pre-defined sensing area 31 (corresponding to the intersects of the conductive columns 13 and the conductive rows 14) to achieve piezoresistance as shown in FIG. 3. The conductive materials in the piezoresistive ink are conductive polymer or nano material with binder for strong attachment to the fabrics. The resistance of the material is adjusted by the concentration of conductive material in the ink. Viscosity of the ink is tuned according to the selected printing/coating method such as spray coating or dispensing. In order to maintain the soft feeling during contact, it is preferred to coat the fabric only in the sensing area 31 instead of soaking the whole fabric with formulated ink. To complete the sensor, the position of the piezoresistive region aligns with the intersects between the conductive columns 13 in the top electrode sheet 20 and the corresponding conductive rows 14 in the bottom electrode sheet 21. The piezoresistive layer 24 need to be optimized to achieve a uniform conduction path between the top electrode sheet 20 and the bottom electrode sheet 21 for each of the sensors on the pressure sensor mat 11.

With a continuous pressure data collected over time through the pressure sensor mat 11, an actuator 40 is provided to adjust the support through a real-time change of pillow shape in response to any significant change in pressure from a preceding time to a present time. The actuator 40 in the present invention comprises one or more airbags made of, for example, but not limited to rubber, forming multiple actuation zones of the pillow. FIG. 4 shows an actuator system 40 including two layers and four zones of airbags, i.e., three zones of airbags on top layers and one zone of airbag on bottom layers. The bottom layer airbag 44 can quickly adjust the height of the pillow, and top layer airbags 41, 42 and 43 can control the relative position of head and neck area, thus adjust the tilting angle between head and neck. Meanwhile, the airbags would be covered with a topping layer such as memory foam or fibrous cover sheet and the pressure sensor mat 11 is positioned on the topping layer and the actuator system 40. The sensors in the pressure sensor mat 11 are utilized to monitor pressurized condition and contact condition of the actuator system 40. In addition, the airbags are designed to lift human body without air leakage and the lifting displacement for each airbag is approximately from 0 mm to 50 mm.

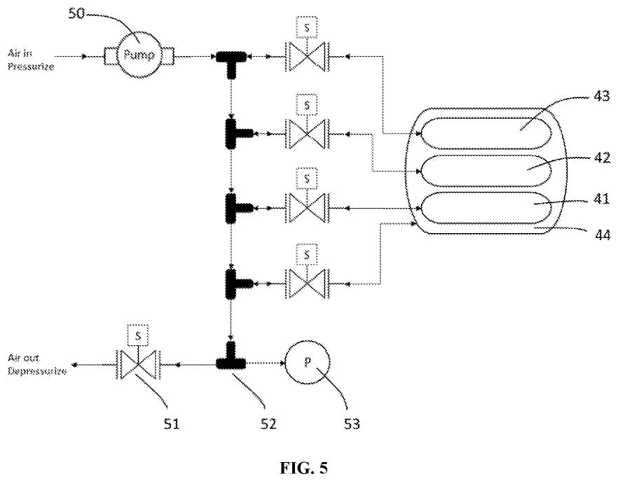

As shown in FIG. 5, the airbags in each of the actuation zones are connected to relay valves 51 and T-connector tube joint 52 through flexible silicone tubes. Together with a micro pump 50 and other necessary components such as internal pressure sensor 53, the airbags can be individually controlled to convert the airbags between an inflated configuration corresponding to an increasing volume of the airbags and a deflated configuration corresponding to a decreasing volume of the airbags. According to the contact pressure distribution, the control unit gives command to relay valves 51 to open or close based on manual or automatic adjustment through algorithm.

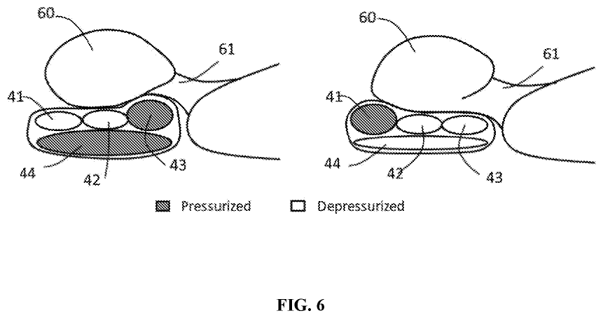

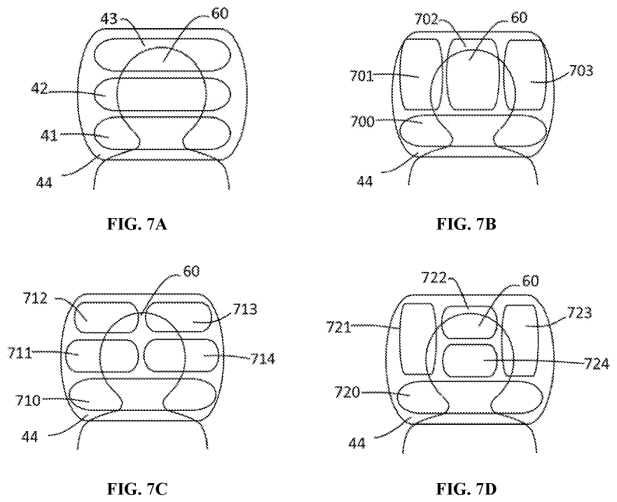

FIG. 6 shows a scenario of sleeping posture adjustment using the reactive pillow in one embodiment of the present invention. When the airbags 44 of the bottom layer are pressurized or depressurized, the height of the pillow can be increased or decreased to meet the comfortableness preference of individual user. When the top layer airbags 41, 42 and 43, are pressurized or depressurized against each other, it can alter the relative height of the front area (near neck) and the back area (near head). Thus, it is able to adjust a tilt angle between head and neck, which could align the neck with spine better so as to improve sleeping quality. Rotation adjustment (from left to right) can be achieved by changing the number of inflated/deflated airbags/zones in the actuator system 40. FIGS. 7B, 7C and 7D respectively show different number of airbags in the actuator system 40 in the various embodiments of the present invention. Once a non-symmetrical pressure pattern is detected, the reactive pillow in the present invention can adjust the height on left and/or right side(s) to keep a person in a balanced sleeping posture. In one embodiment, the airbags/zones are connected with each other and can be controlled by the same pressure valve. In other embodiment, each of the airbags/zones can be controlled by a separate pressure valve.

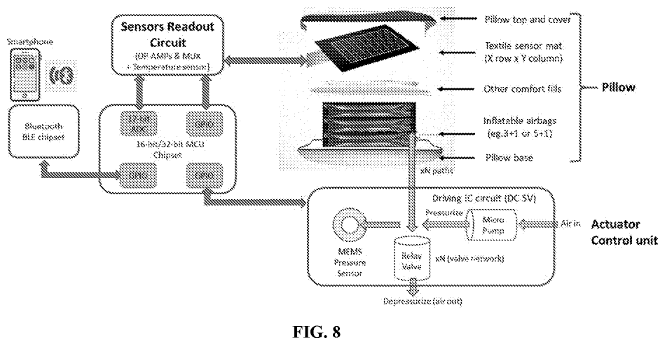

FIG. 8 is a block diagram showing the hardware for the reactive pillow in the present invention. The system unit is powered by standard USB port of power of 5V with 0.5 A. The hardware system consists of a set of ICs (Op-AMP and MUX) circuit to read the voltage output signal of voltage-current converter amplifier probed to each sensor on the pressure sensor mat 11. Meanwhile, there is a MEMS IC to read the inner pressure from the actuator system 40. All these voltage signals will then be converted to digital data by multiple channels of ADC converter built-in in the 16-bit/32-bit MCU IC. A set of ICs (motor driver) circuit is also required to drive the micro pump 50 and relay valves 51.

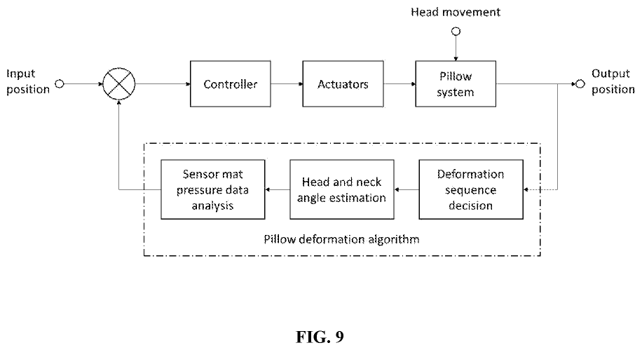

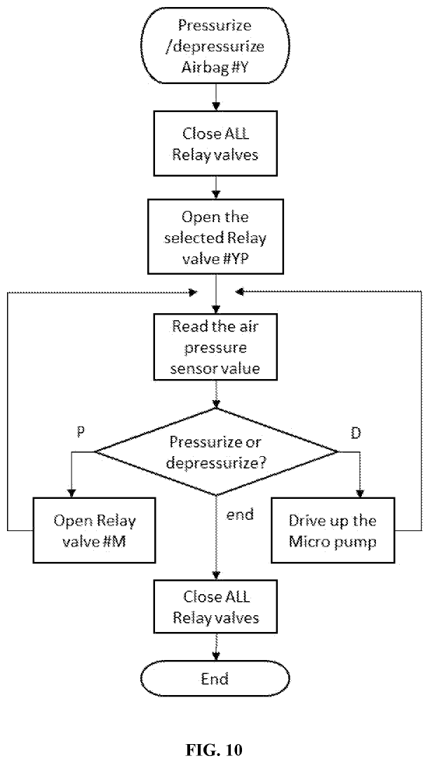

All data process and control are handled by the software program installed on the 16-bit/32-bit MCU IC. Furthermore, user data will be recorded and sent to a portable device where there is a user interface to show and/or even change the setting or preference of the present pillow such as a program or an application on a smartphone, and the MCU IC and the portable device can be communicated wirelessly such as via Bluetooth in the presence of a Bluetooth module. User may therefore change the setting or preference of the pillow by the portable device through the corresponding program or application wirelessly. The program may include at least three parts. A first part is a data acquisition module acquiring data from different sensing mechanisms including the sensor array, inner pressure sensor of corresponding airbags and the temperature sensor thereof. Sampling rate of data acquisition from these sensing mechanisms may be limited by corresponding sensor array size and/or response time of the sensor(s) in the array. A second part is an output module including drivers of the micro pump 50 and relay valves 51 with a speed control. A third part is a feedback control system module with a pillow deformation algorithm, as shown in FIG. 9, so as to adjust tilt angle between head and neck, and learn from comfortable user experience fed to the system to make deformation decision based on the real-time pressure data received from the sensor mat through the shape transition of pillow. More specifically, the sensor mat will firstly identify whether the user is in side sleeping position or back sleeping position by analyzing the pressure distribution of head and neck regions, followed by subjecting to the pillow deformation algorithm to allow user to manually select pillow height based on their preference. In one embodiment of the present invention, inflation of the airbags can be adjusted to approximately 80 to 100% in a region close to the neck of the subject when the sensor mat identifies he/she is in side sleeping position. In another embodiment, inflation of the airbags can be adjusted to approximately 20 to 40% in a region close to the neck when the subject is identified to be in a back-sleeping position. The comfortableness with respect to each sleeping position can be selected by user based on their preference within a workable range of adjustable inflation of the airbags. Referring to FIG. 10, it also shows in a flow diagram how pressurization and depressurization of the airbag is decided and executed according to one embodiment of the present invention.

Therefore, the reactive pillow in the present invention mainly has the following features and advantages: (1) The sensor array for pressure detection is flexible and conforms to 3-dimensional surface for simultaneous pressure sensing and monitoring over large area. The fabric pressure sensor mat is positioned on top surface of pillow for accurate pressure monitoring yet with improved comfortableness. The electrodes and piezoresistive layer fabricated by or including conductive and semi-conductive yarns provide high reliability. (2) The actuator system can transform pillow shape according to the signal from the pressure sensor mat in real time. (3) Control electronics and software establish communication between the reactive pillow and the mobile device, which enables continuous data recording wirelessly.

Although the invention has been described in terms of certain embodiments, other embodiments apparent to those of ordinary skill in the art are also within the scope of this invention. Accordingly, the scope of the invention is intended to be defined only by the claims which follow.

1. A reactive pillow comprising:
a pressure sensor mat comprising
a first electrode fabric layer having a plurality of first conductive portions and a plurality of first non-conductive portions being interlaced with the first non-conductive portions,
a second electrode fabric layer having a plurality of second conductive portions and a plurality of second non-conductive portions being interlaced with the second non-conductive portions, and
a piezoresistive fabric layer having a sheet resistance of at least 50K ohm/square, the piezoresistive fabric layer being configured to engage with the first electrode fabric layer and the second electrode fabric;
an actuator comprising a plurality of first airbags and a second airbag, the first airbags positioned on the second airbag, each of the airbags having an inflated configuration corresponding to increase the volume of the airbags and a deflated configuration corresponding to decrease the volume of the airbags; and
a controller communicating with the pressure sensor mat and the actuator communicating with the controller to actuate the controller to convert the airbags between the inflated configuration and the deflated configuration thereof.

2. The reactive pillow of claim 1, wherein the first conductive portions and the second conductive portions are substantially aligned perpendicularly.

3. The reactive pillow of claim 1, wherein the first and second conductive portions are woven or knitted fabric made of or comprising conductive yarns.

4. The reactive pillow of claim 1, wherein the first and second non-conductive portions are woven or knitted fabric made of or comprising non-conductive yarns.

5. The reactive pillow of claim 1, wherein each of the first and second conductive portions has a width of approximately 3 mm to 10 mm.

6. The reactive pillow of claim 1, wherein each of the first and second non-conductive has a width of approximately 5 mm to 50 mm.

7. The reactive pillow of claim 1, wherein the piezoresistive fabric layer is a woven or knitted fabric made of or comprising semi-conductive yarns.

8. The reactive pillow of claim 7, wherein the piezoresistive fabric layer further comprises a non-conductive fabric and a plurality of filling portions incorporated with a piezoresistive ink.

9. The reactive pillow of claim 8, wherein the piezoresistive ink comprises a polymer, a conductive material and a solvent.

10. The reactive pillow of claim 9, wherein the polymer has a concentration approximately from 1% to 10% by weight; the conductive material has a concentration approximately from 0.1% to 2% by weight; and the solvent has a concentration approximately from 90% to 95% by weight.

11. The reactive pillow of claim 8, wherein the sheet resistance of the filling portions is at least 50K ohm/square.

12. The reactive pillow of claim 8, wherein the filling portions have one or more shapes being selected from circle, square, and rectangle, with a width approximately from 5 mm to 15 mm and a space approximately from 3 mm to 50 mm.

13. The reactive pillow of claim 1, wherein the actuator further comprises at least one micro pump, at least one tube and at least one valve, and the actuator is configured to convert the airbags between the inflated configuration and the deflated configuration thereof.

14. The reactive pillow of claim 1, wherein the first airbags are configured to convert between inflated configuration and the deflated configuration thereof such that the relative volume corresponding to the left, right, top bottom of the pillow can be adapted.

15. The reactive pillow of claim 14, wherein the first airbags comprises at least three airbags.

16. The reactive pillow of claim 1, wherein the second airbag is configured to convert between inflated configuration and the deflated configuration such that relative volume corresponding to the height of the pillow can be adapted.

17. The reactive pillow of claim 1, wherein the reactive pillow further comprises a Bluetooth module configured to communicate with a user terminal.

18. A method for fabricating a reactive pillow, comprising:
providing a pressure sensor mat comprising a first electrode fabric layer having a plurality of first conductive portions and a plurality of first non-conductive portions where the first conductive portions are interlaced with the first non-conductive portions, a second electrode fabric layer having a plurality of second conductive portions and a plurality of second non-conductive portions where the second conductive portions are interlaced with the second non-conductive portions and a piezoresistive fabric layer having a sheet resistance of at least 50K ohm/square, the piezoresistive fabric layer configured to engage with the first electrode fabric layer and the second electrode fabric;
providing an actuator comprising a plurality of first airbags and a second airbag, the first airbags positioned on the second airbag, each of the airbags having an inflated configuration corresponding to increase the volume of the airbags and a deflated configuration corresponding to decrease the volume of the airbags; and
providing a controller communicating with the pressure sensor mat and the actuator communicating with the controller to actuate the controller to convert the airbags between the inflated configuration and the deflated configuration;
wherein the first conductive portions and the second conductive portions are aligned perpendicularly.

19. The method of claim 18, wherein the first and second conductive portions have a width approximately from 3 mm to 10 mm, and the first and second non-conductive have a width approximately from 5 mm to 50 mm.

20. The method of claim 18, wherein the piezoresistive ink comprises a polymer with a concentration approximately from 1% to 10% by weight, a conductive material with a concentration approximately from 0.1% to 2% by weight and a solvent with a concentration approximately from 90% to 95% by weight.
